(19) United States
(12) Patent Application Publication
(10) Pub. No.: US 2004/0261291 A1
(43) Pub. Date: Dec. 30, 2004

(54) Shoe sole having a non-flat surface for accommodating the non-flat undersurface of a foot resting on the sole

(76) Inventor: Sang Kyun Paek, Inchun-City (KR)

Correspondence Address: LADAS & PARRY LLP, 224 SOUTH MICHIGAN AVENUE, SUITE 1200, CHICAGO, IL, 60604, US

(21) Appl. No.: 10/722,885

(22) Filed: Nov. 26, 2003

(30) Foreign Application Priority Data
Dec. 12, 2002 (KR) 20-2002-0037189

(51) Int. Cl. A43B 13/00; A61F 5/14; A43B 23/00; A43B 13/38

(52) U.S. Cl. 36/25 R; 36/174; 36/180; 36/43

(57) ABSTRACT

A sole of a shoe has a recessed or depressed portion to receive a pair of sesamoid bones protruding the undersurface of a foot wearing the shoe. The shoe sole may be an attached part of the shoe or a piece separable from the shoe. The depth of the depressed portion is about 2 mm. A sole of a shoe may have an aperture, instead of a recessed portion, to receive a pair of sesamoid bones.

### BACKGROUND OF THE INVENTION

1. Field of the Invention

This application relates generally to a sole of a shoe and more particularly to a sole of a shoe that has a non-flat surface for accommodating the non-flat undersurface of a human foot that rests on the sole of the shoe when the shoe is worn.

2. Description of the Prior Art

Generally, a conventional sole of a shoe has a flat surface although the undersurface of a foot that wears the shoe and that rests on the sole of the shoe is not flat.

In particular, a pair of bones known as the sesamoid bones in the anatomy of a human foot protrudes underneath the main toe of a foot. An example of the pair of sesamoid bones is shown in FIG. 5 as a pair of the semamoid bones 4 protrudes underneath the main toe 5.

As shown in FIG. 5, this pair of the sesamoid bones 4 is a part of the non-flat undersurface structure of a foot. In particular, when the non-flat undersurface of a foot rests on the flat conventional sole 3 of a shoe 1, 6, the protruding sesamoid bones 4 cause discomforts to the person wearing the shoe 1, 6.

As the protruding sesamoid bones 4 rub against the flat sole 3 of the shoe 1, 6, this causes the foot to become tired easily and may cause pain. Further, the protruding sesamoid bones 4 may cause the foot wearing the conventional flat-sole shoe 1, 3, 6 to lean and bend to either left or right side of the human body depending on the foot as the person wearing the shoe 1, 3, 6 moves by taking the steps for walking or running.

Accordingly, there is a need for providing a sole of a shoe that solves the problems associated with the conventional shoe 1, 6 having a flat-sole 3.

### SUMMARY OF THE INVENTION

Against this backdrop, embodiment(s) of the present invention have been developed.

A sole of a shoe has a depressed or recessed portion to receive a pair of sesamoid bones protruding the undersurface of a foot wearing the shoe. The shoe sole may be an attached part of the shoe or a piece separable from the shoe. The depth of the depressed portion is about 2 mm. A sole of a shoe may have an aperture, instead of a recessed portion, to receive a pair of sesamoid bones.

These and various other features as well as advantages which characterize the present invention will be apparent from a reading of the following detailed description and a review of the associated drawings.

### BRIEF DESCRIPTION OF THE DRAWINGS

FIG. 1 is a top view showing the upper-half of a sole of a shoe according to an embodiment of the present invention.

FIG. 2 is a cross-sectional view showing the cross-section along the line AA of the upper half of a sole of a shoe of FIG. 1.

FIG. 3 is a cross-sectional view showing the cross-section along the line BB of the upper half of a sole of a shoe of FIG. 1.

FIG. 4 is a cross-sectional view showing a cross-section of a sole of a shoe along with a cross-section of a foot wearing the shoe according to an embodiment of the present invention.

FIG. 5 is a cross-sectional view showing a cross-section of a conventional sole of a shoe along with a cross-section of a foot wearing the conventional shoe having the flat-surface sole.

### DETAILED DESCRIPTION

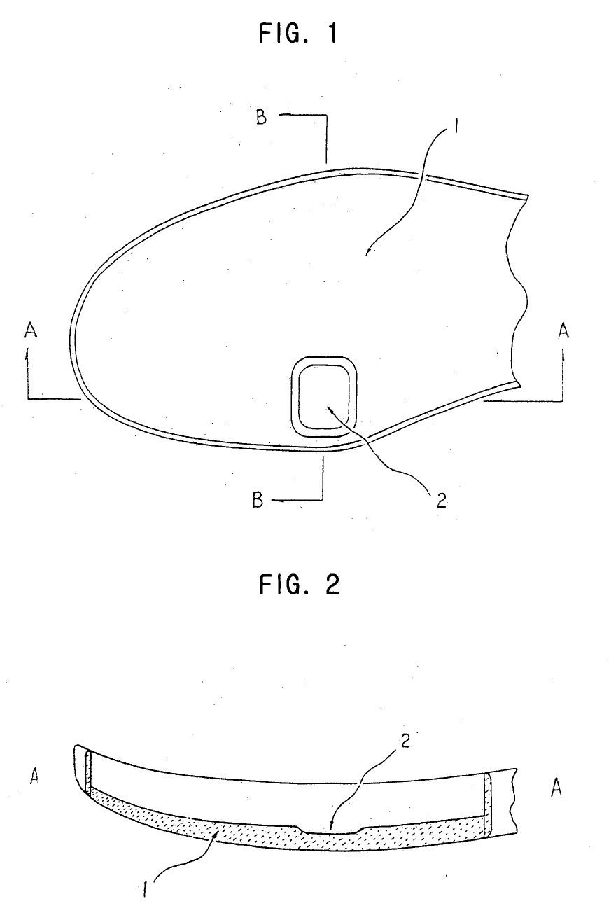

FIG. 1 is a top view showing the upper half of a sole 1 of a shoe according to an embodiment of the present invention. As shown in FIG. 1, a recessed or depressed portion 2 of the sole 1 has a depression to accommodate the protruding pair of sesamoid bones 4 (FIG. 4).

FIG. 2 shows the depression depth of the recessed or depressed portion 2 shown along the line AA of the sole 1 of FIG. 1. Generally, the depth of the recessed or depressed portion 2 is about 2 mm; however, other depths are possible depending on the structure of the sesamoid bones so that the depressed portion 2 can be tailored for the comfort of an individual wearing the shoe.

Further, it is noted that the recessed portion 2 can be formed by simply creating a hole or an aperture through the sole 1 to receive the protruding pair of sesamoid bones 4 and thereby to provide comfort.

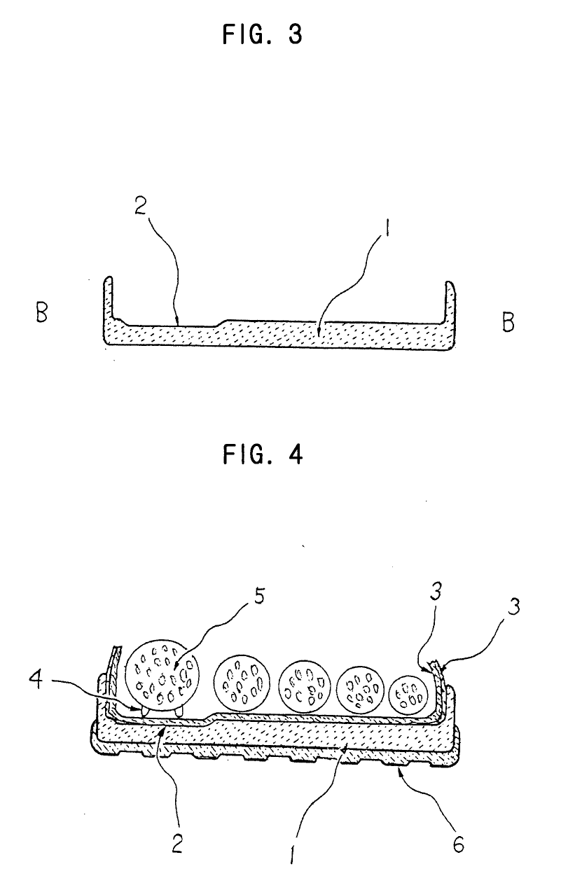

FIG. 3 shows the depression depth of the depressed portion 2 shown along the line BB of the upper half of a sole 1 of a shoe of FIG. 1.

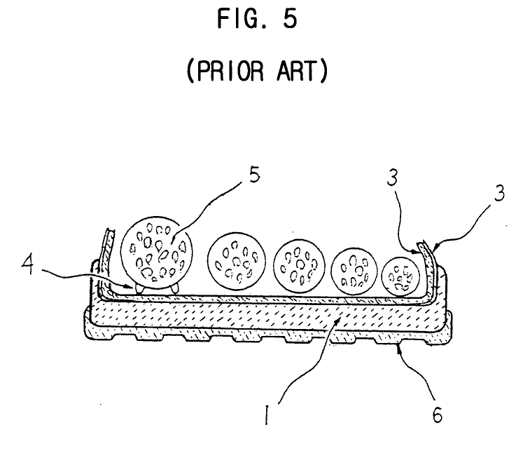

FIG. 4 is a cross-sectional view showing a cross-section of a sole of a shoe along with a cross-section of a foot wearing the shoe according to an embodiment of the present invention. As shown in FIG. 4, the protruding sesamoid bones 4 of the main toe 5 is received in the depressed portion 2 of the sole 3 so that the comfort is provided to the person wearing the shoe and the problems caused by wearing the flat sole shoe (as shown in FIG. 5) are solved.

The sole 3 of a shoe 1, 6 or the shoe 1, 6 itself including the sole 3 is typically made from a synthetic material. The sole 3 including the recessed portion 2 is often made by a molding process. Nevertheless, it is noted that the sole 3 of a shoe 1, 6 according to an embodiment of the present invention can be made from various materials, whether they are synthetic or natural, and by various manufacturing processes including the molding process. Further, it is noted that the sole 3 of a shoe 1, 6 according to an embodiment of the present invention may be a separate piece, for example, an insert or a shoe liner, separable from the shoe 1, 6 or an attached part of the shoe 1, 3, 6.

It will be clear that the present invention is well adapted to attain the ends and advantages mentioned as well as those inherent therein. While a various embodiments including the presently preferred one has been described for purposes of this disclosure, various changes and modifications may be made, which are well within the scope of the present invention. Numerous other changes may be made which will readily suggest themselves to those skilled in the art and which are encompassed in the spirit of the invention disclosed and as defined in the appended claims.

What is claimed is:

1. A sole of a shoe having a recessed portion in an area of the sole that corresponds to the location of a pair of sesamoid bones protruding undersurface of a foot wearing the shoe for receiving the sesamoid bones in the recessed portion to provide comfort to the person wearing the shoe.

2. The shoe sole of claim 1, wherein the sole is an attached part of the shoe.

3. The shoe sole of claim 2, wherein the depth of the recessed portion is about 2 mm.

4. The shoe sole of claim 1, wherein the sole is separable from the shoe.

5. The shoe sole of claim 4, wherein the depth of the recessed portion is about 2 mm.

6. A sole of a shoe having an aperture formed in an area of the sole that corresponds to the location of a pair of sesamoid bones protruding undersurface of a foot wearing the shoe for receiving the sesamoid bones in the aperture to provide comfort to the person wearing the shoe.

7. The shoe sole of claim 6, wherein the sole is an attached part of the shoe.

8. The shoe sole of claim 6, wherein the sole is separable from the shoe.
